(19) United States
(12) Patent Application Publication
(10) Pub. No.: US 2010/0316660 A1
(43) Pub. Date: Dec. 16, 2010

(54) EDIBLE PRODUCT HAVING AN IMMUNOSTIMULATING EFFECT

(76) Inventors: Johanna Alida Van Adrichem, Vlaardingen (NL); Wilhelmina Martina Blom, Vlaardingen (NL); Yvonne Elisabeth Maria Dommels, Vlaardingen (NL); Jean Hypolites Koek, Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/808,812

(22) PCT Filed: Dec. 2, 2008
(86) PCT No.: PCT/EP2008/066579

§ 371 Date: Jun. 17, 2010

(30) Foreign Application Priority Data
Dec. 20, 2007 (EP) 07123803.4

(51) Int. Cl. A61K 39/00

(52) U.S. Cl. 424/184.1

(57) ABSTRACT

There is provided an edible product having an immunostimulating effect, said product comprising polysaccharides obtainable from the Alliaceae family of the perennial flowering plants. Also provided is a process for preparing such an edible product and composition comprising from 0.0001 to 25% by weight of polysaccharides obtainable from the Alliaceae family and having an immunostimulating effect

### TECHNICAL FIELD

The present invention relates to an edible product. More in particular, it relates to an edible product having an immunostimulating effect, especially an edible product comprising immunostimulating polysaccharides obtainable from the Alliaceae family of the perennial flowering plants.

### BACKGROUND ART

Most adults suffer two to five colds per year, and infants and pre-school children have an average of four to eight. The upper respiratory tract (URT) infections, like common colds and flu, are together with gastro-intestinal (GI) infections the most important reasons of absenteeism at work or school. In a lifetime of 75 years, we suffer on average from over 200 episodes of common cold. This means that if each cold lasts for five to seven days we spend around three years of our life coughing and sneezing with colds. The need and interest of a consumer in “self prevention” and “self treatment” of these acute infections are therefore high.

A recent interest in the field of functional food ingredients is the use of immunomodulators for enhancing host defence responses, for instance, to provide more protection against the common cold. An important part of the host defence response is the innate immune system. The innate arm of the immune system is a rapidly activated first line of defence against pathogens. It involves amongst others phagocytic and natural killer (NK) cells. Phagocytic cells such as neutrophils, monocytes and macrophages can generate reactive oxygen species (ROS) to kill pathogens such as fungi, bacteria and virus-infected cells. NK cells can kill target cells that have lost or express insufficient amounts of MHC class I, a frequent event in tumor- or virus-infected cells.

Some edible products or food products are known to have immunostimulating properties. For example, US-A-2005/0002962 discloses a melanin preparation of botanicals such as Echinacea, American ginseng, black walnut, green tea, Parthenium integrifolium, Korean ginseng, alfalfa sprouts, ginger, goldenseal, red clover, dandelion, black cohosh, licorice, chamomile, milk thistle, alfalfa, horsetail, astragalus, gotu kola, feverfew, valerian, hawthorn, rosemary, saw palmetto, ephedra, pau d'arco, ginkgo, garlic, St. John's wort, Agaricus bisporus (common mushroom), Agaricus bisporus brown strain (portabella mushroom), Lentinus edodes (shiitake mushroom) or Boletus edulis (porcini mushroom) as an immune stimulatory composition.

Josling (2001) discloses that an allicin-containing garlic supplement can prevent healthy human volunteers against the common cold (Josling (2001) Preventing the common cold with a garlic supplement: a double-blind, placebo controlled survey. Advances in Therapy 18: 189193). The main bioactive substances present in Allium vegetables, such as garlic and onion are organosulfur compounds, such as allicin, accounting for 65 to 75% of the total organosulfur compounds in garlic.

There is a constant need for new or alternative food products having such immunostimulating properties. It is therefore an object of the present invention to provide such edible products. It is a further object of the invention to provide a process for the preparation of such food products having such immunostimulating properties.

It was surprisingly found that the object can be achieved by the edible product of the invention, which comprises immunostimulating polysaccharides obtainable from the Alliaceae family of the perennial flowering plants.

Scientific literature discloses that a cold water extract of green onion (Allium fistulosum) or garlic (Allium sativum) both have anti-inflammatory activity. This was based on the inhibitory effect of nitric oxide (NO) production by lipopolysaccharide (LPS)-activated macrophages (Tsai T H, Tsai P J, Ho S C (2005) Antioxidant and anti-inflammatory activities of several commonly used spices). However, the immune stimulating properties of polysaccharides from the Alliaceae family of the perennial flowering plants has never been disclosed.

### SUMMARY OF THE INVENTION

According to a first aspect of the present invention, there is provided an edible product having an immunostimulating effect, said product comprising immunostimulating polysaccharides obtainable from plants of the Alliaceae family of the perennial flowering plants.

According to a second aspect of the invention, there is provided a process for preparing the edible product according to the invention.

According to a third aspect of the invention, there is provided a composition comprising from 0.0001 to 25% by weight of polysaccharides having an immunostimulating effect and having an average degree of polymerization >3.

### DETAILED DESCRIPTION OF THE INVENTION

According to a first aspect, the present invention relates to an edible product comprising polysaccharides which are responsible for the immunostimulating effect. The polysaccharides used in the present product may be derived from plants belonging to the Alliaceae family of the perennial flowering plants.

Alliaceae is a family of herbaceous perennial flowering plants. They are monocots, part of the order Asparagales. The family has been widely but not universally recognized; in the past, the plants involved were often treated as belonging to the family Liliaceae, and still are by some botanists.

The most important genus is Allium, which includes several important food plants, including onions (Allium cepa), chives (A. schoenoprasum), garlic (A. sativum and A. scordoprasum), and leeks (A. porrum).

According to the present invention, the immunostimulating polysaccharides are preferably derived from plants of the genus Allium. It is especially preferred if the plant is selected from the group consisting of Allium including onions (Allium cepa), chives (A. schoenoprasum), garlic (A. sativum and A. scordoprasum), and leeks (A. porrum). Onions and garlic are especially preferred. The immunostimulating polysaccharides may be isolated from the plants of the Alliaceae family by a process which involves harvesting the plant, cutting up the plant, especially the bulbs, homogenising or mashing the plant material and (freeze) drying it to form a dry powder. Subsequently, the dried powder is extracted with hot, preferably boiling, water and the extract is (freeze) dried. A polysaccharide enriched extract may be prepared by using two additional washing steps with 85% ethanol at 80° C.

The immunostimulating polysaccharides can be characterised by their average degree of polymerization which is >3, preferably >5, more preferably >7, up to 1,000.

The edible product according to the present invention may take any physical form. In particular, it may be a liquid or a spreadable, spoonable solid or a food supplement.

Preferably the product is a liquid product. The edible product may suitably take the form of e.g. a soup, a beverage, a spread, a dressing, a dessert or a mayonnaise. More preferably, the edible product is a beverage, a dessert or a spread. More preferably, the edible product is a beverage or a spread, especially a spread in the form of an oil-in-water emulsion. The term “spread” as used herein encompasses spreadable products such as margarine, spreadable cheese based products and processed cheese. Most preferably, the present product is a beverage. Such a beverage typically contains at least 60 wt. % water and 0-20 wt. % of dispersed fat. Preferably, such beverage contains at least 70 wt. % water and 0-10 wt. % of dispersed fat.

By the term “immunostimulating” as used herein is meant that the activity or capacity of the immune system to defend itself against pathogens such as fungi, bacteria, viruses, protozoa, parasites or proteins is increased. Immunostimulation thus contributes to an enhanced natural defence of the human body. Several assays can be used to identify components that could modify immunity. The present inventors chose the use of phagocytic and natural killer (NK) cells to aid the identification of immunostimulating compounds as these cells are part of the innate immune system, which is a rapidly activated non-specific first line of defence against pathogens.

Phagocytic cells such as neutrophils, monocytes and macrophages can generate reactive oxygen species (ROS) to kill pathogens such as fungi and bacteria. The effect of ingredients on phagocytosis activity can be measured ex vivo with fresh blood of healthy human volunteers after incubation with FITC-labelled E. coli bacteria. The percentage of phagocytosing cells in the granulocyte population can be determined by flow cytometry. The results are typically normalized to the effect of lipo-polysaccharide (LPS), which is a well known potent immunostimulating reference compound. In the present invention a normalized % phagocytosing granulocytes >40% is regarded as a significant immune stimulating effect.

NK cells can kill target cells that have lost or express insufficient amounts of MHC class I, a frequent event in tumor- or virus-infected cells. The effect of ingredients on NK cell activity can ex vivo be measured with peripheral blood mononuclear cells (PBMC) isolated from fresh blood of healthy human volunteers. After pre-incubation of the PBMCs with the ingredient, pre-labelled K562 target cells are usually added and after subsequent incubation, propidium iodide can be added for detection of dead cells. The percentage of dead target cells can be determined with flow cytometry. The results are typically normalized to the effect of interleukin-2 (IL-2), which is a well known potent NK cell stimulating reference compound. In the present invention a normalized % NK cell activity >17% is regarded as a significant immune stimulating effect.

The edible product according to the invention preferably includes additional nutrients, vitamins and minerals to deliver healthy nutrition. Suitable vitamins and minerals, include but are not limited to Vitamin A, Vitamin D, Vitamin E, Vitamin C, Thiamin, Riboflavin, Niacin, Vitamin B6, folate, Vitamin B12 (cyanocobalamin), Biotin, Pantothenic acid, Calcium, Phosphorous, Potassium, Iron, Zinc, Copper, Iodine, Selenium, Sodium, Magnesium, Manganese, molybdenum, vitamin K, chromium and mixtures thereof. The preferred ingredients to deliver vitamins and minerals include but are not limited to potassium phosphate, calcium phosphate, magnesium oxide, magnesium phosphate ascorbic acid, sodium ascorbate, vitamin E acetate, niacinamide, ferric orthophosphate, calcium pantothenate, zinc oxide, zinc gluconate, vitamin A palmitate, pyridoxine hydrochloride, riboflavin, thiamin mononitrate, biotin, folic acid, chromium chloride, potassium iodide, sodium molybdate, sodium selenate, phytonadone (vitamin K), cholecalciferol (vitamin D3), manganese sulfate and mixtures thereof. Preferably, the inventive product contains at least 10% or more of the recommended daily amount (“RDA”) of the vitamins and minerals.

The inventive products may further include meat, fish, meat and fish extracts, fruit, dried fruit, fruit concentrates, fruit extracts, fruit juices, tea (e.g. green tea), vegetables, vegetable extracts and concentrates, nuts, nut extracts, chocolate, bread, vinegar, salt, pepper, cocoa powder, herbs (e.g. parsley), herb extracts, spices (e.g. cinnamon), spice extracts, emulsifiers, acidity regulators (e.g. phosphoric, malic, citric, tartaric acids and salts thereof), flavonoids, preservatives (e.g. lactic acid, EDTA, tocopherols, sodium benzoate), colors (e.g. beta carotene, lycopene, caramel, carmine red), fibers (e.g. soy), leavening agents (e.g., sodium bicarbonate), pectin, citric acid, yeast, salt, glycerin, and mixtures thereof.

According to a second aspect of the invention, the edible product according to the invention is prepared by adding the immunostimulating polysaccharides to a food product, e.g. during the production process.

A further aspect of the present invention is a process to obtain the polysaccharides having an immunostimulating effect. Plant based polysaccharides consist of large insoluble polymers, like cell wall components, small soluble oligosaccharides, like monomers (e.g. glucose) and dimers (e.g. cellobiose), and large soluble polysaccharides. Especially from the latter an immunomodulating response is expected as they are large enough to provoke a reaction from the immune system and solubility is a requirement for interaction. In order to enrich plant based material in soluble polysaccharides, the small oligosaccharides may to be removed. This is usually done (Shiomi, N., 1992. New Phytologist 122, pp. 421-432.) by a repeated warm alcohol (85%) wash step as the small oligosaccharides have some solubility in the aqueous alcohol while the polymers are insoluble. Subsequently a hot water extraction is applied to the residue to isolate the water soluble polymer and separate it from insoluble polysaccharides. In this step also other water insoluble components are removed. In order to check the success of carbohydrate isolation an overall content of carbohydrates is determined using the Dubois method (Dubois M. et al, (1956) Colorimetric method for determination of sugars and related substances, Analytical Chemistry, 28(3), 350-356). A first rough insight in the success of removal of small oligosaccharides is obtained by the average degree of polymerization which is determined by comparing the analysis result on carbohydrate reducing end groups (DNSA method) with the total carbohydrate content determined by the Dubois method. Successful removal of small oligosaccharides (e.g. mono and disaccharides) would give a high average DP value (e.g. at least higher than 2). A more accurate way is to determine the molecular weight distribution of the enriched extract by size exclusion chromatography.

A further aspect of the invention is a composition comprising from 0.0001 to 25% by weight of polysaccharides obtainable from plants of the Alliaceae family and having an immunostimulating effect, said polysaccharides having an average degree of polymerization >3, preferably >5, more preferably >7 up to 1,000.

This invention will now be described in more detail by means of the following Examples. In the Figures:

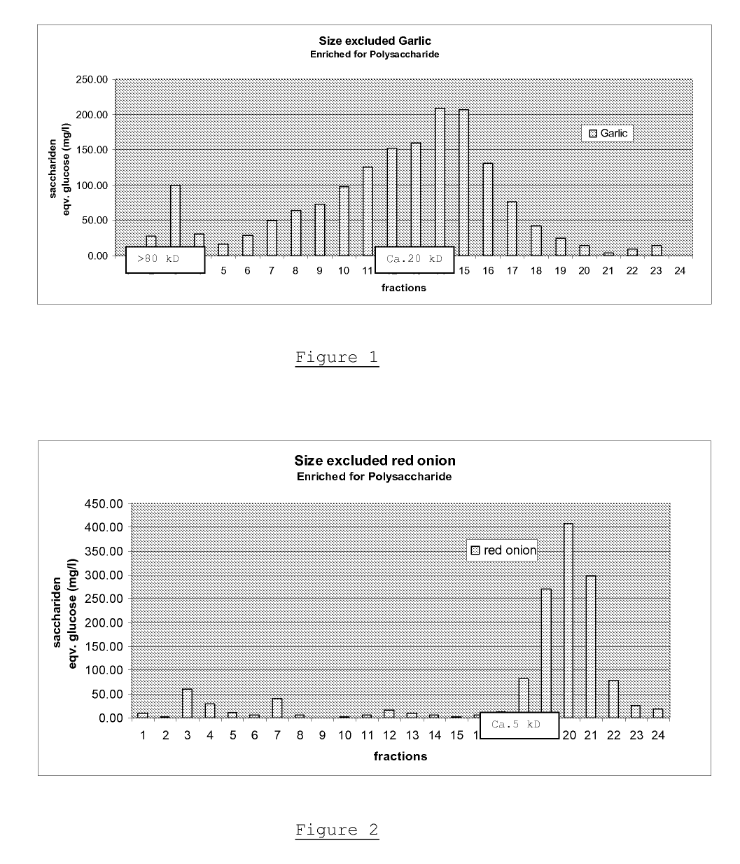

FIG. 1 shows a size exclusion chromatogram of 5 g/l polysaccharide-enriched extract of Alliacea (Garlic, A),

FIG. 2 shows a size exclusion chromatogram of 5 g/l polysaccharide-enriched extract of Red onion (B),

FIG. 3 shows the effect of a polysaccharide-enriched extract of garlic on natural killer (NK) cell activity of human PBMCs,

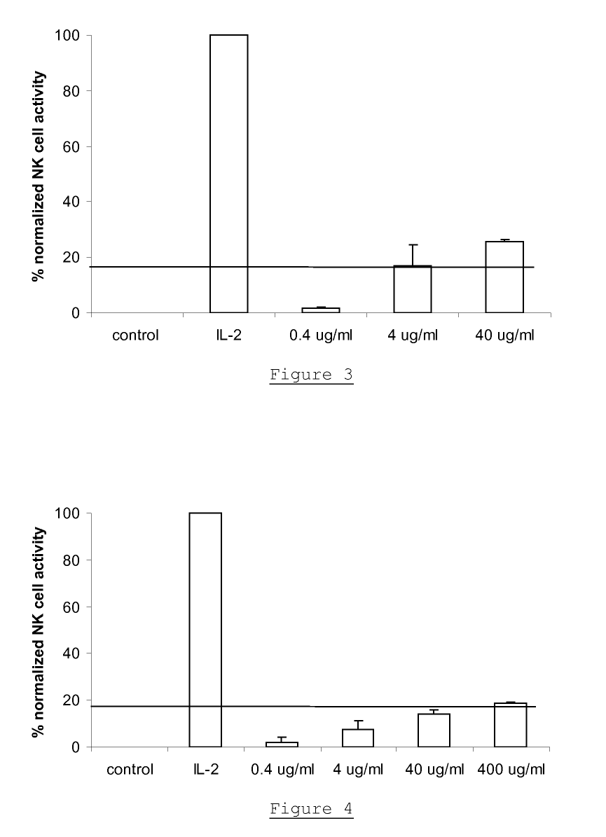

FIG. 4 shows the effect of a polysaccharide-enriched extract of red onion on natural killer (NK) cell activity of human PBMCs,

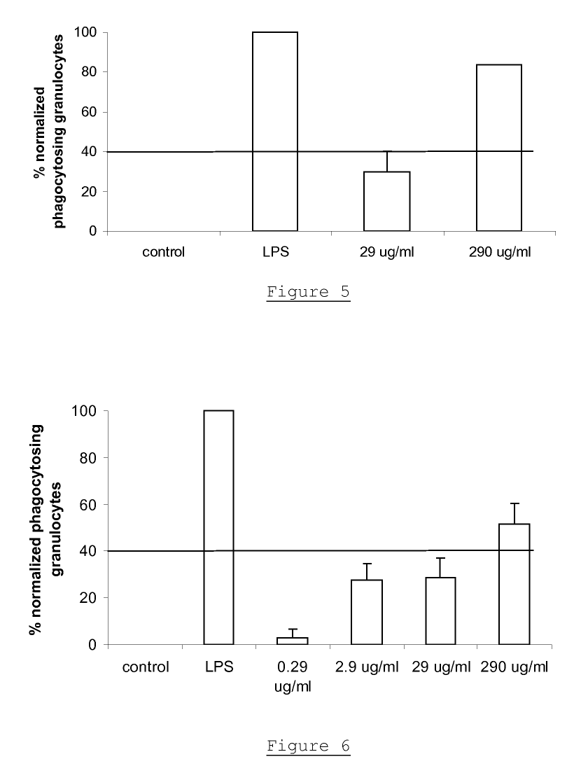

FIG. 5 shows the Effect of a polysaccharide-enriched extract of garlic on phagocytosis activity of fresh human blood samples, and

FIG. 6 shows the effect of a polysaccharide-enriched extract of red onion on phagocytosis activity of fresh human blood samples.

### EXAMPLE 1

### Extraction Procedure to Obtain an Edible Composition Comprising a Polysaccharide-Enriched Extract of Alliaceae (A: Garlic, B: Red Onion)

The polysaccharide-enriched extract of Alliaceae was obtained as follows: 25 g of either garlic powder (obtained from Starwest Botanical Item#205230-51) or red onion (obtained from Euroma “vriesdroog rode ui”) was washed 2 times with 200 ml of 85% ethanol (VWR Prolabo) in water for 2.5 hours at 80° C. and 1 time with 200 ml of 85% ethanol in water for 1.5 hours at 80° C. After decanting the ethanol, the pellet was dried overnight in a fuming cabinet. The polysaccharides were extracted by adding 200 ml of MilliQ water and boiling for 3 hours. After centrifugation at 2,000 g for 20 minutes at room temperature (RT), the pellet was re-suspended in 200 ml of MilliQ water and boiled again for 3 hours. The supernatant of the first and second extraction were collected, lyophilized and stored at room temperature. From the polysaccharide enriched lyophilized extracts a 2% (m/m) suspension in LAL water was made and autoclaved for 15 minutes at 121° C. The suspension was centrifuged at 2000 g for 30 minutes at RT. The supernatant was filtered through a 0.2 μm filter, divided in small portions and stored at −20° C., until use in the immune assays.

### EXAMPLE 2

### Analytical Characterization of the Edible Composition Comprising a Polysaccharide-Enriched Extract of Alliaceae (A: Garlic, B: Red Onion)

Quantification of Carbohydrates

The total amount of carbohydrates in the sample was measured according to the method of Dubois (vide supra). 50 μl of test sample or standard curve sample (0-0.3 g/l glucose) was mixed with 20 μl of 6 g/l resorcinol (Aldrich) and 90 μl pure H₂SO₄ p.a. (Merck). After an incubation of 20 minutes at 80° C. the extinction was measured at 450 nm. The amount of carbohydrates was calculated via linear regression of the calibration curve with glucose.

Determination of the Monosaccharide Composition

A 10 g/l solution of an edible composition comprising a polysaccharide-enriched extract of garlic powder (A) or red onion (B) was hydrolyzed in 2 M HCL to obtain monosaccharides. The hydrolysis was done by addition of 0.2 ml 37% hydrochloric acid to 1 ml solution giving a final concentration of 2 M HCl, followed by thoroughly mixing and an incubation of 6 hours at 95° C. in a pre-heated water bath. After this incubation, the solution was cooled down to RT and centrifuged for 10 minutes at 15600×g. The pH of the supernatant was adjusted to a pH between 3 and 7 with 10 M NaOH, filter-sterilized (0.45 μm) and 0.5 ml was placed in a 1 ml HPLC vial for analysis (LC-10AT, Shimadzu, Japan). Both monosaccharide samples (10 μl) were injected. For calibration, monosaccharides (10 μl solutions of 1 g/l concentrations) were injected (all were purchased at Sigma-Aldrich) using an auto injector SIL-10AD, Shimadzu, Japan. Sulphuric acid (5 mM, pH 2.0) was used as an eluent and an Aminex HPX-87H (300×7.8 mm) column was used at a temperature of 65° C. and a flow-rate of 0.6 ml/min. Dual determination of refractive index (RID-10A, Shimadzu, Japan) and UV 220 nm and 280 nm (SPD-10A, Shimadzu, Japan) was used. Based on the calibration with the monosaccharide's the monosaccharide composition present in the hydrolyzed samples was determined.

Determination of the Degree of Polymerization

The molecular amount of polysaccharides in the samples was measured according to the method of Bernfeld (Bernfeld et al (1955) Amylase alpha and beta. Methods in Enzymology, 1, 149-158). A 3,5-dinitrosalicylic acid (DNSA) reagent was made by dissolving one gram DNSA in 20 ml 2 M NaOH and 50 ml water at 60° C. After obtaining a clear solution, 30 g potassium sodium tartrate was added and the volume was adjusted to 100 ml. 150 μl of test sample or standard curve sample (0-25 mmol/l glucose) was mixed with 150 μl DNSA reagent. Three times 50 μl of the sample DNSA reagent mixture was added to a 96-well plate. After heating for 30 minutes at 80° C. and cooling to room temperature, 100 μl of water was added and the absorbance at 540 nm was measured against a blank. The molecular amount of polysaccharides was calculated via linear regression of the calibration curve with glucose.

By dividing the amount of carbohydrates (g/kg sample) with the molecular amount (mol/kg sample), the average molecular weight (g/mol) was determined.

Determination of the Molecular Weight Distribution

The distribution of the molecular weight was done with preparative size exclusion chromatography performed with Äcta explorer (GE Healthcare, Sweden) with a P900 pump and a UV900 detector. The Sephacryl 5200 HR was packed in an XK100/1.6 column according to the manufacturers instructions (GE Healthcare note 52-2086-00), using D-PBS without CaCl₂ and MgCl₂ (GIBCO BRL) as a buffer, pH=7.0.

The column was equilibrated with D-PBS buffer without CaCl₂ and MgCl₂ at a flow rate of 1 ml/min. 2 ml samples or standard was loaded at a flow rate of 0.5 ml/min and the isocratic elution was done at 0.5 ml/min.

For the dextran standards of 80 kD, 50 kD, 25 kD, 12.5 kD, 5 kD and 1 kD, a concentration of 1 g/l was used. For the polysaccharide-enriched extract of garlic (A) or red onion (B) 5 g/l in buffer was used. Fractions of 4.8 ml were collected between 65 ml and 180 ml during elution of the samples. Of the fractions the amount of carbohydrates was measured according to the method of Dubois.

Characterization of an Edible Composition Comprising a Polysaccharide-Enriched Extract of Garlic (A) or Red Onion (B)

The total amount of carbohydrates, the monosaccharide composition, the degree of polymerization and the protein content of an edible composition comprising a polysaccharide-enriched extract of garlic (A) or red onion (B) are shown in table 1. The molecular weight distribution of the polysaccharide-enriched extract of garlic (A) or red onion (B) is shown in FIGS. 1 and 2. The composition of extract A is characterized by carbohydrates over 95%, different monosaccharides, mainly glucose and negligible level of protein <5% and an average degree of polymerization of >200. The composition of extract B, the PS-enriched extract of red onion is characterized by carbohydrates in the range between 64% and 87%, also a high level of glucose and an unidentified sugar as in extract A but in different quantities, total protein in the range between 19 and 24% and an average degree of polymerization of 6.9. From the size exclusion chromatography it is also clear that the molecular weight distribution of the garlic extract peaks at a higher MW (between 50 and 12.5 kD) compared to the red onion extract (between 5 kD and 12 kD). Both extracts also contain small amount of high molecular weight oligosaccharide's (>80 kD).

TABLE 1
Characterization of an edible composition comprising a
polysaccharide-enriched extract of garlic (A) or red onion (B)

| | Polysaccharide- | |
|---|---|---|
| | enriched | Polysaccharide- |
| | extract of | enriched extract |
| | garlic (A) | of red onion (B) |
| Carbohydrate (CH) | >95% | 64%-87% |
| (weight % dry matter) | | |
| Monosaccharide | | |
| composition (% of | | |
| total CH) | | |
| Glucuronic acid | n.d. | n.d. |
| Galacturonic acid | 1.1 | 8.5 |
| Glucose | 37.1 | 38.2 |
| Galactose/Xylose/ | 3.1 | 10.2 |
| Fructose | | |
| Rhamnose | 0.4 | 0.7 |
| Fucose | 0.5 | n.d. |
| Arabinose | 0.7 | 0.8 |
| unknown | 57.1 | 41.6 |
| Degree of | 200 | 6.9 |
| polymerization | | |
| Protein | <5% | 19%-24% |
| (weight % dry matter) | | |

### EXAMPLE 3

### Natural Killer (NK) Cell Stimulating Effect of a Polysaccharide-Enriched Extract of Alliaceae (A: Garlic, B: Red Onion)

Determination of Natural Killer (NK) Cell Activity

Fresh blood was obtained from healthy volunteers in sodium heparin tubes and peripheral blood mononuclear cells (PBMC) were isolated from the blood by density gradient centrifugation using Ficoll-Paque. PBMC were counted and resuspended in tissue culture medium RPMI 1640 Complete at a concentration of 5×10⁶ cells/ml and stored at 4° C. until use as effector cells in the flow cytometric cytotoxicity assay. The erythromyelocytic leukemia cell line K562, a NK sensitive cell line, was used as target cells. K562 cells were washed with PBS, counted and resuspended in PBS at a concentration of 5×10⁶ cells/ml before labeling with the dye carboxyfluorescein diacetate succinimidyl ester (CFDA-SE) at 1 μg/ml for 30 min at 5% CO₂, 37° C. After labeling, cells were washed with PBS and resuspended in RPMI Complete at a concentration of 10×10⁴ cells/ml and stored at 4° C. in the dark until use.

PBMCs were pre-incubated with the ingredient in triplicate for 30 minutes at 37° C. (5% CO₂). Control incubations consisted of PBS (=basal level of NK cell activity) or 800 IU/ml interleukin-2 (IL-2) (=positive control sample) (mean±sd in triplicate). After pre-incubation with the ingredient, target cells were added (ratio effector:target cells=25:1) and incubated for 120 minutes at 37° C. (5% CO₂). After incubation, cells were placed on ice for 1-5 minutes after addition of propidium iodide for detection of dead cells. Natural killer cell activity was measured on the Coulter FC500 MPL flow cytometer (Beckman Coulter, Miami, Fla., USA). Data of at least 1000 target cells were collected and analyzed using the EXPO 32 program. The percentage of dead target cells was determined. The results were normalized to the effect of IL-2.

Modulation of NK Cell Activity by Alliaceae

An edible composition comprising a polysaccharide-enriched extract of garlic (A) was tested at three different concentrations in the NK cell activity assay (0.4-4-40 μg/ml). The results of this experiment are shown in FIG. 3. An edible composition comprising a polysaccharide-extract of red onion (B) was tested at four different concentrations in the NK cell activity assay (0.4-4-40-400 μg/ml). The results of this experiment are shown in FIG. 4.

Results represent means and the standard deviation of one experiment with each sample performed in duplicate. The NK cell activity is reported as percentage of maximal stimulation by interleukin-2 whereby a % normalized NK activity >17 is regarded as an immune stimulatory effect (=arbitrary threshold). The results show that a polysaccharide-enriched extract of red onion (B) stimulates NK cell activity of PBMC isolated from human blood samples at a concentration of 400 μg/ml. A polysaccharide-enriched extract of garlic (A) is already effective at a concentration of 4 and 40 μg/ml.

### EXAMPLE 4

### Phagocytosis Stimulating Effect of a Polysaccharide-Enriched Extract of Alliaceae (A: Garlic, B: Red Onion)

Determination of Phagocytosis Activity

Phagocytosis activity was measured with the Phagotest® kit of Orpegen Pharma (Heidelberg, Germany) using an adjusted protocol. In more detail:

Fresh blood was obtained from healthy human volunteers in sodium heparin vacutainers (BD biosciences). 30 ul of whole blood and 5 ul of the ingredient were pre-incubated in duplicate for 30 minutes in a polypropylene 96-well plate at 37° C. in a water bath. Control incubations consisted of PBS (=basal phagocytosis activity) or 100 ng/mL E. coli-lipopolysaccharide (LPS) (=positive control sample) (mean±sd in triplicate). After the pre-incubation step, 10 μl of FITC-labeled E. coli (white blood cell to E. coli ratio of 25:1) was added. This incubation at 37° C. was stopped after 6.5 minutes by adding 50 ul of ice-cold quencher solution. The cells were washed three times by adding 230 μl of ice-cold wash-buffer and centrifugation for 3 min at 300 g (4° C.). The erythrocytes were lysed by addition of 290 μl of lysis buffer. After incubation in the dark for 20 minutes at room temperature, the cells were centrifuged for 5 min at 300 g (4° C.). Cells were resuspended in 150 μl of wash-buffer and stained with propidium iodide. Analysis was performed by flow cytometry (Coulter FC500 MPL flow cytometer, Beckman Coulter Nederland BV, Mijdrecht). Leukocytes were gated into monocyte and granulocyte populations according to the FSC/SSC profile. The percentage of phagocytosing cells in the granulocyte population was determined. The results were normalized to the effect of lipopolysaccharide (LPS).

Modulation of Phagocytosis Activity by Alliaceae

An edible composition comprising a polysaccharide-enriched extract of garlic (A) was tested at two different concentrations in the phagocytosis activity assay (29-290 μg/ml). The results of this experiment are shown in FIG. 5. An edible composition comprising a polysaccharide-extract of red onion (B) was tested at four different concentrations in the phagocytosis activity assay (0.29-2.9-29-290 μg/ml). The results of this experiment are shown in FIG. 6. Results represent means and the standard deviation of one experiment with each sample performed in duplicate. The phagocytosis activity is reported as percent of maximal stimulation by LPS whereby a % normalized phagocytosing granulocytes >40% is regarded as an immune stimulatory effect (=arbitrary threshold). The results show that an edible composition comprising a polysaccharide-enriched extract of garlic or a polysaccharide-enriched extract of red onion both stimulate phagocytosis activity of granulocytes isolated from fresh human blood samples at a concentration of 290 μg/ml.

1. Edible product having an immunostimulating effect, said product comprising immunostimulating polysaccharides obtainable from the Alliaceae family of the perennial flowering plants, wherein the polysaccharides have an average degree of polymerization >3.

2. Edible product according to claim 1, wherein the polysaccharides are obtainable from plants of the Allium genus.

3. Edible product according to claim 1, wherein the plant is selected from the group consisting of Allium including onions (Allium cepa), chives (A. schoenoprasum), garlic (A. sativum and A. scordoprasum), and leeks (A. porrum).

4. Edible product according to claim 1, wherein the polysaccharides are obtainable from garlic or onion.

5. Edible product according to claim 1, wherein the immunostimulating polysaccharides are obtainable by a process comprising the steps of harvesting the plants, cutting up the plants, especially the bulbs, and preparing a hot-water extract of the insoluble material.

6. Edible product according to claim 5, wherein the hot-water extract is enriched by additional washing steps of the water-insoluble material.

7. Edible product according to claim 1, in the form of a liquid, such as a soup, a beverage, a spread, a dressing, a dessert or a mayonnaise.

8. Process for preparing an edible product according to claim 1, comprising the steps of
(a) harvesting the plants,
(b) cutting up the plants, especially the bulbs,
(c) preparing a hot-water extract of the insoluble material and
(d) adding said extract to a edible product.

9. Process for preparing an edible product according to claim 8, wherein the process comprises one or more additional washing steps.

10. Composition comprising from 0.0001 to 25% by weight of immunostimulating polysaccharides obtainable from plants of the Alliaceae family and having an immunostimulating effect, said polysaccharides having an average degree of polymerization >3.

11. The edible product according to claim 1, wherein the average degree of polymerization is >7 to 1000.

12. The composition of claim 10 wherein the average degree of polymerization is >7 to 1000.
